(19) United States
(12) Patent Application Publication
(10) Pub. No.: US 2024/0000514 A1
(43) Pub. Date: Jan. 4, 2024

(54) SURGICAL PLANNING FOR BONE DEFORMITY OR SHAPE CORRECTION

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Branislav JARAMAZ, Pittsburgh, PA (US); Constantinos NIKOU, Monroeville, PA (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 18/265,088

(22) PCT Filed: Jan. 6, 2022
(86) PCT No.: PCT/US22/11384

§ 371 Date: Jun. 2, 2023

Related U.S. Application Data
(60) Provisional application No. 63/135,145, filed on Jan. 8, 2021.

(51) Int. Cl. A61B 34/10; A61B 34/20; G16H 50/70; G16H 30/40; A61B 34/30

(52) U.S. Cl. CPC A61B 34/10; A61B 34/20; G16H 50/70; G16H 30/40; A61B 34/30; A61B 2034/105; A61B 2034/2055

(57) ABSTRACT

The present disclosure provides a machine learning model to model a normal version of a bone from an abnormal version of the bone. The machine learning model can be trained with a training set including abnormal bone images and corresponding normalized, or post-operative, bone images. The abnormal bone image and the inferred normal bone image can be used to plan a surgery to correct the abnormal bone with a surgical navigation system.

### CROSS-REFERENCE TO RELATED APPLICATION

This application claims the benefit of U.S. Provisional Application Ser. No. 63/135,145 filed Jan. 8, 2021, entitled “Surgical Planning for Bone Deformity or Shape Correction,” which application is incorporated herein by reference in its entirety.

### TECHNICAL FIELD

This disclosure relates generally to computer-aided orthopedic surgery apparatuses and methods to address acetabular impingement. Particularly, this disclosure relates to determining what material needs to be removed during orthopedic surgery to alter an abnormal bone.

### BACKGROUND

The use of computers, robotics, and imaging are increasing used to aid orthopedic surgery. For example, computer-aided navigation and robotics systems can be used to guide orthopedic surgical procedures. As a specific example, a precision freehand sculptor (PFS) employs a robotic surgery system to assist the surgeon in accurately shaping a bone. In interventions such as correction of acetabular impingement, computer-aided surgery techniques have been used to improve the accuracy and reliability of the surgery. Orthopedic surgery guided by images has also been found useful in preplanning and guiding the correct anatomical position of displaced bone fragments in fractures, allowing a good fixation by osteosynthesis.

Femoral acetabular impingement (FAI) is a condition characterized by abnormal contact between the proximal femur and rim of the acetabulum. In particular, impingement occurs when the femoral head or neck rubs abnormally or does not have full range of motion in the acetabular socket. It is increasingly suspected that FAI is one of the major causes of hip osteoarthritis. Cam impingement and pincer impingement are two major classes of FAI. Cam impingement results from pathologic contact between an abnormally shaped femoral head and neck with a morphologically normal acetabulum. The femoral neck is malformed such that the hip range of motion is restricted and the deformity on the neck causes the femur and acetabular rim to impinge on each other. This can result in irritation of the impinging tissues and is suspected as one of the main mechanisms for development of hip osteoarthritis. Pincer impingement is the result of contact between an abnormal acetabular rim and a typically normal femoral head and neck junction. This pathologic contact is the result of abnormal excess growth of anterior acetabular cup. This results in decreased joint clearance and repetitive contact between the femoral neck and acetabulum, leading to degeneration of the anterosuperior labrum.

Orthopedic surgery to address femoral acetabular impingement is typically an arthroscopic procedure. Due to the limited accessibility of the bone by the surgeon, an accurate surgical plan is desired to determine what material needs removed. This need is magnified when the surgical plan will be used to assist in controlling a robotic arm during the procedure.

### BRIEF SUMMARY

Thus, it would be beneficial to precisely model a “normal” version of the patient's femur so the surgeon can model the anatomy that needs removed. In particular, using machine learning (ML) models as described herein provides that actual anatomy can be modeled more accurately than is possible through statistical modeling. It is with this in mind that the present disclosure is presented.

In one feature, a method includes receiving, at a computing device, a representation of an abnormal bone, inferring a representation of a normalized bone associated with the abnormal bone based on executing a machine learning (ML) model at the computing device with the representation of the abnormal bone as input to the ML model, identifying a region of deformity on the abnormal bone based on the representation of the normalized bone, and generating a surgical plan for altering the abnormal bone based on the region of deformity.

The method may also include partitioning the abnormal bone into a plurality of segments, partitioning the normalized bone into a plurality of segments, and identifying the region of deformity from the segments of the abnormal bone.

The method may also include extracting a first plurality of anatomical features from the abnormal bone, extracting a second plurality of anatomical features from the normalized bone, and comparing the first plurality of features to the second plurality of features to identify the region of deformity.

The method may also include where the ML model includes a convolutional neural network (CNN).

The method may also include where the ML model is trained with a data set that includes a plurality of images of pathological bones and for each one of the plurality of images of the pathological bones, an associated image of a non-pathological bone. Other technical features may be readily apparent to one skilled in the art from the following figures, descriptions, and claims.

In one feature, a non-transitory computer-readable storage medium, the computer-readable storage medium including instructions that when executed by a computer, cause the computer to receive, at a computing device, a representation of an abnormal bone, infer a representation of a normalized bone associated with the abnormal bone based on executing a machine learning (ML) model at the computing device with the representation of the abnormal bone as input to the ML model, identify a region of deformity on the abnormal bone based on the representation of the normalized bone, and generate a surgical plan for altering the abnormal bone based on the region of deformity.

The computer-readable storage medium may also include instructions that cause the computing device to partition the abnormal bone into a plurality of segments, partition the normalized bone into a plurality of segments, and identify from the segments of the abnormal bone the region of deformity.

The computer-readable storage medium may also include instructions that cause the computing device to extract a first plurality of anatomical features from the abnormal bone, extract a second plurality of anatomical features from the normalized bone, compare the first plurality of features to the second plurality of features to identify the region of deformity.

The computer-readable storage medium may also include where the ML model includes a convolutional neural network (CNN).

The computer-readable storage medium may also include where the ML model is trained with a data set that includes a plurality of images of pathological bones and for each one of the plurality of images of the pathological bones, an associated image of a non-pathological bone. Other technical features may be readily apparent to one skilled in the art from the following figures, descriptions, and claims.

In one feature, a computing apparatus includes a processor. The computing apparatus also includes a memory storing instructions that, when executed by the processor, configure the apparatus to receive, at a computing device, a representation of an abnormal bone, infer a representation of a normalized bone associated with the abnormal bone based on executing a machine learning (ML) model at the computing device with the representation of the abnormal bone as input to the ML model, identify a region of deformity on the abnormal bone based on the representation of the normalized bone, and generate a surgical plan for altering the abnormal bone based on the region of deformity.

The computing apparatus may also include instructions that cause the computing apparatus to partition the abnormal bone into a plurality of segments, partition the normalized bone into a plurality of segments, and identify from the segments of the abnormal bone the region of deformity.

The computing apparatus may also include instructions that cause the computing apparatus to extract a first plurality of anatomical features from the abnormal bone, extract a second plurality of anatomical features from the normalized bone, compare the first plurality of features to the second plurality of features to identify the region of deformity.

The computing apparatus may also include where the ML model includes a convolutional neural network (CNN).

The computing apparatus may also include where the ML model is trained with a data set that includes a plurality of images of pathological bones and for each one of the plurality of images of the pathological bones, an associated image of a non-pathological bone. Other technical features may be readily apparent to one skilled in the art from the following figures, descriptions, and claims.

The method may also include where at least one of the associated images of the non-pathological bone is of a post-operative pathological bone.

The method may also include where the plurality of images of the pathological bones are classified as having at least one of the same bone type, the same gender assigned at birth, the same ethnicity, or the same age range.

The method may also include where the bone type is a femur.

The method may also include includes generating control signals for a surgical tool of a surgical navigation system based on the surgical plan.

The computer-readable storage medium may also include where at least one of the associated images of the non-pathological bone is of a post-operative pathological bone.

The computer-readable storage medium may also include where the plurality of images of the pathological bones are classified as having at least one of the same bone type, the same gender assigned at birth, the same ethnicity, or the same age range.

The computer-readable storage medium may also include where the bone type is a femur.

The computer-readable storage medium may also include includes generate control signals for a surgical tool of a surgical navigation system based on the surgical plan.

The computing apparatus may also include where at least one of the associated images of the non-pathological bone is of a post-operative pathological bone.

The computing apparatus may also include where the plurality of images of the pathological bones are classified as having at least one of the same bone type, the same gender assigned at birth, the same ethnicity, or the same age range.

The computing apparatus may also include where the bone type is a femur.

The computing apparatus may also include includes generate control signals for a surgical tool of a surgical navigation system based on the surgical plan.

A surgical navigation system, including a surgical cutting tool, and the computing apparatus described above coupled to the surgical cutting tool, where the control signals are for the surgical cutting tool.

Further features and advantages of at least some of the embodiments of the present disclosure, as well as the structure and operation of various embodiments of the present disclosure, are described in detail below with reference to the accompanying drawings. Other technical features may be readily apparent to one skilled in the art from the following figures, descriptions, and claims.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

It is noted, the drawings are not necessarily to scale. The drawings are merely representations, not intended to portray specific parameters of the disclosure. The drawings are intended to depict example embodiments of the disclosure, and therefore are not considered as limiting in scope. In the drawings, like numbering represents like elements.

Furthermore, certain elements in some of the figures may be omitted for illustrative clarity. The cross-sectional views may be in the form of “slices”, or “near-sighted” cross-sectional views, omitting certain background lines otherwise visible in a “true” cross-sectional view, for illustrative clarity. Furthermore, for clarity, some reference numbers may be omitted in certain drawings.

FIG. 1 illustrates surgical planning system 100, in accordance with embodiment(s) of the present disclosure.

FIG. 2A illustrate a 3D image 200a, in accordance with embodiment(s) of the present disclosure.

FIG. 2B illustrates a 3D image 200b, in accordance with embodiment(s) of the present disclosure.

FIG. 3A illustrates a 2D image 300a, in accordance with embodiment(s) of the present disclosure.

FIG. 3B illustrates a 2D image 300b, in accordance with embodiment(s) of the present disclosure.

FIG. 3C illustrates a 2D image 300c, in accordance with embodiment(s) of the present disclosure.

FIG. 4 illustrates a logic flow 400, in accordance with embodiment(s) of the present disclosure.

FIG. 5 illustrates a logic flow 500, in accordance with embodiment(s) of the present disclosure.

FIG. 6 illustrates a system 600, in accordance with embodiment(s) of the present disclosure.

FIG. 7 illustrates a computer-readable storage medium 700, in accordance with embodiment(s) of the present disclosure.

FIG. 8 illustrates a robotic surgical system 800, in accordance with embodiment(s) of the present disclosure.

### DETAILED DESCRIPTION

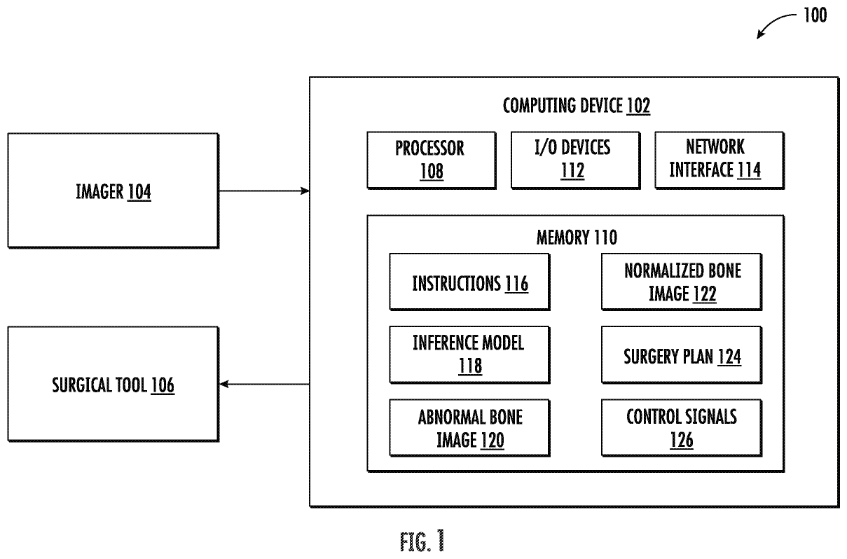

FIG. 1 illustrates a surgical planning system 100, in accordance with non-limiting example(s) of the present disclosure. In general, surgical planning system 100 is a system for planning a surgery on an abnormal bone. In some embodiments, surgical planning system 100 is a system for planning and carrying out a surgery on an abnormal bone. Surgical planning system 100 includes a computing device 102. Optionally, surgical planning system 100 includes imager 104 and surgical tool 106. In an example, computing device 102 can receive an image of an abnormal bone (e.g., abnormal bone image 120, or the like) from imager 104, generate a surgical plan for modifying the abnormal bone (e.g., surgery plan 124, or the like), and control the operation of surgical tool 106 (e.g., via control signals 126, or the like) to alter the abnormal bone based on the surgical plan, such as by surgically removing an excess portion from the abnormal bone.

Imager 104 can be any of a variety of bone imaging devices, such as, for example, an X-ray imaging device, a fluoroscopy imaging device, an ultrasound imaging device, a computed tomography (CT) imaging device, a magnetic resonance (MR) imaging device, a positron emission tomography (PET) imaging device, a single-photon emission computed tomography (SPECT) imaging device, or an arthrogram. Imager 104 can generate information elements, or data, including indications of abnormal bone image 120. Computing device 102 is communicatively coupled to imager 104 and can receive the data including the indications of abnormal bone image 120 from imager 104. In general, abnormal bone image 120 can include indications of shape data and/or appearance data of an abnormal bone. Shape data can include landmarks, surfaces, and boundaries of three-dimensional objections. Appearance data can include both geometric characteristics and intensity information of the abnormal bone. With some examples, abnormal bone image 120 can be constructed from two-dimensional (2D) or three-dimensional (3D) images of the abnormal bone. In some embodiments, abnormal bone image 120 can be a medical image. The term “image” is used herein for clarity of presentation and to imply that abnormal bone image 120 represents the structure and anatomy of the bone. However, it is to be appreciated that the term “image” is not to be limiting. That is, abnormal bone image 120 may not be an image as conventionally used, or rather, an image viewable and interpretable by a human. For example, abnormal bone image 120 can be a point cloud, a parametric model, or other morphological description of the anatomy of the abnormal bone. Furthermore, abnormal bone image 120 can be a single image, a series of images, or an arthrogram. With some examples, computing device 102 can generate abnormal bone image (e.g., morphological description, or the like) from a conventional image or series of conventional images. Examples are not limited in this context.

Examples of the abnormal bone can include a femur, an acetabulum, or any other bone in a body to be altered by surgical planning system 100. In general, surgical tool 106 can be a surgical navigation system or a medical robotic system. In particular, surgical tool 106 can be a robotic device adapted to assist and/or perform an orthopedic surgery to revise the abnormal bone, such as, for example, surgery to revise a femur to correct FAI. As part of the surgical navigation system, surgical tool 106 can include a bone tracking device, a surgical tool tracking device, a surgical tool positioning device, or the like.

Computing device 102 can be any of a variety of computing devices. In some embodiments, computing device 102 can be incorporated into and/or implemented by a console of surgical tool 106. With some embodiments, computing device 102 can be a workstation or server communicatively coupled to imager 104 and/or surgical tool 106. With still other embodiments, computing device 102 can be provided by a cloud-based computing device, such as, by a computing as a service system accessibly over a network (e.g., the Internet, an intranet, a wide area network, or the like). Computing device 102 can include processor 108, memory 110, input and/or output (I/O) devices 112, and network interface 114.

The processor 108 may include circuitry or processor logic, such as, for example, any of a variety of commercial processors. In some examples, processor 108 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 108 may include graphics processing portions and may include dedicated memory, multiple-threaded processing and/or some other parallel processing capability. In some examples, the processor 108 may be an application specific integrated circuit (ASIC) or a field programmable integrated circuit (FPGA).

The memory 110 may include logic, a portion of which includes arrays of integrated circuits, forming non-volatile memory to persistently store data or a combination of non-volatile memory and volatile memory. It is to be appreciated, that the memory 110 may be based on any of a variety of technologies. In particular, the arrays of integrated circuits included in memory 110 may be arranged to form one or more types of memory, such as, for example, dynamic random access memory (DRAM), NAND memory, NOR memory, or the like.

I/O devices 112 can be any of a variety of devices to receive input and/or provide output. For example, I/O devices 112 can include, a keyboard, a mouse, a joystick, a foot pedal, a display, a touch enabled display, a haptic feedback device, an LED, or the like.

Network interface 114 can include logic and/or features to support a communication interface. For example, network interface 114 may include one or more interfaces that operate according to various communication protocols or standards to communicate over direct or network communication links. Direct communications may occur via use of communication protocols or standards described in one or more industry standards (including progenies and variants). For example, network interface 114 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, network interface 114 can include logic and/or features to enable communication over a variety of wired or wireless network standards (e.g., 802.11 communication standards). For example, network interface 114 may be arranged to support wired communication protocols or standards, such as, Ethernet, or the like. As another example, network interface 114 may be arranged to support wireless communication protocols or standards, such as, for example, Wi-Fi, Bluetooth, ZigBee, LTE, 5G, or the like.

Memory 110 can include instructions 116, inference model 118, abnormal bone image 120, normalized bone image 122, surgery plan 124, and control signals 126. During operation, processor 108 can execute instructions 116 to cause computing device 102 to receive abnormal bone image 120 from imager 104. Processor 108 can further execute instructions 116 and/or inference model 118 to generate normalized bone image 122 from inference model 118. Normalized bone image 122 can be data comprising a normal or “normalized” bone which has a comparable anatomy to the abnormal bone to be altered by surgical tool 106.

Inference model 118 can be any of a variety of machine learning models. In particular, inference model 118 can be an image classification model, such as, a neural network (NN), a convolutional neural network (CNN), a random forest model, or the like. Inference model 118 is arranged to infer normalized bone image 122 from abnormal bone image 120. Said differently, inference model 118 can infer an image of a normal bone or normalized bone which has an anatomical origin comparable to the abnormal bone represented by abnormal bone image 120. As used herein, a normal or normalized bone is a bone lacking abnormalities or a bone, which had abnormalities that have been removed. For example, for an FAI surgery, the surgeon's goal or target when performing the surgery is often not a “normal” femur. Instead, the bone is resected in an artificial way, and thus, the ideal anatomy or the normalized bone can be non-pathological. Thus, the term normal or normalized is used when referring to the bone post-modification or post-surgery. This normal or normalized bone is represented by normalized bone image 122. Likewise, the term image as used in normalized bone image 122 can be a conventional medical image, a point cloud, a parametric model, or other morphological description or representation of the normalized bone.

Processor 108 can execute instructions 116 to generate surgery plan 124 from normalized bone image 122 and abnormal bone image 120. In general, surgery plan 124 can include a “plan” for altering a portion of the abnormal bone represented by abnormal bone image 120 to conform to the normalized bone represented by normalized bone image 122. In general, processor 108 can execute instructions 116 to determine a level of disconformity between the bone represented in abnormal bone image 120 and the bone represented in normalized bone image 122. This disconformity can be used as a basis for surgical planning or generating a surgical plan. Said differently, processor 108 can execute instructions 116 to generate a plan including indications of revisions or resections to make to the abnormal bone during a surgery.

With some examples, processor 108 can execute instructions 116 to cause I/O devices 112 to present information in audio, visual, or other multi-media formats to assist a surgeon during the process of creating and evaluating surgery plan 124. Examples of the presentation formats include sound, dialog, text, or 2D or 3D graphs. The presentation may also include visual animations such as real-time 3D representations of the abnormal bone image 120, normalized bone image 122, surgery plan 124, or the like. In certain examples, the visual animations can be color-coded to further assist the surgeon to visualize the one or more regions on the abnormal bone that needs to be altered according to surgery plan 124. Furthermore, processor 108 can execute instructions 116 to receive, via I/O devices 112, input to accept or modify surgery plan 124.

Processor 108 can further execute instructions 116 to generate control signals 126 comprising indications of actions, movements, operations, or the like to control surgical tool 106 to implement or carry out the surgery plan 124. Additionally, processor 108 can execute instructions 116 to cause control signals 126 to be communicated to surgical tool 106 (e.g., via network interface 114, or the like) during an orthopedic surgery.

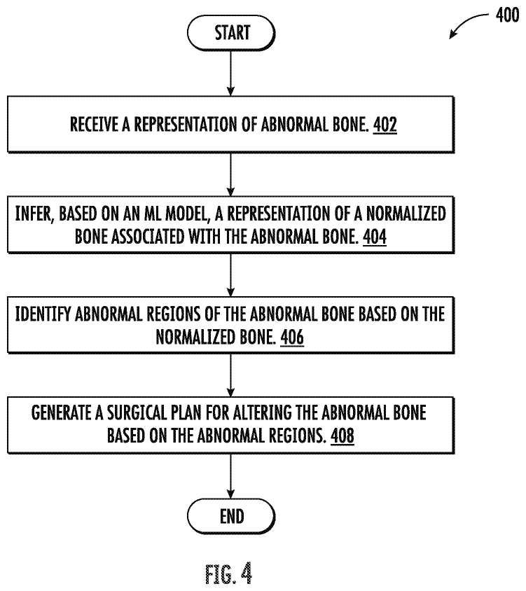

The above is described in greater detail below, such as, for example, in conjunction with logic flow 400 from FIG. 4. With some examples, surgical planning system 100 can be provided with just computing device 102. That is, surgical planning system 100 can include computing device 102 and a user of surgical planning system 100 can provide imager 104 and surgical tool 106 that are compatible with computing device 102. In another example, surgical planning system 100 can include just instructions 116 and inference model 118 and a user can supply abnormal bone image 120, which can be executed by a comparable computing system (e.g., a cloud computing service, or the like) to generate a surgical plan as described herein.

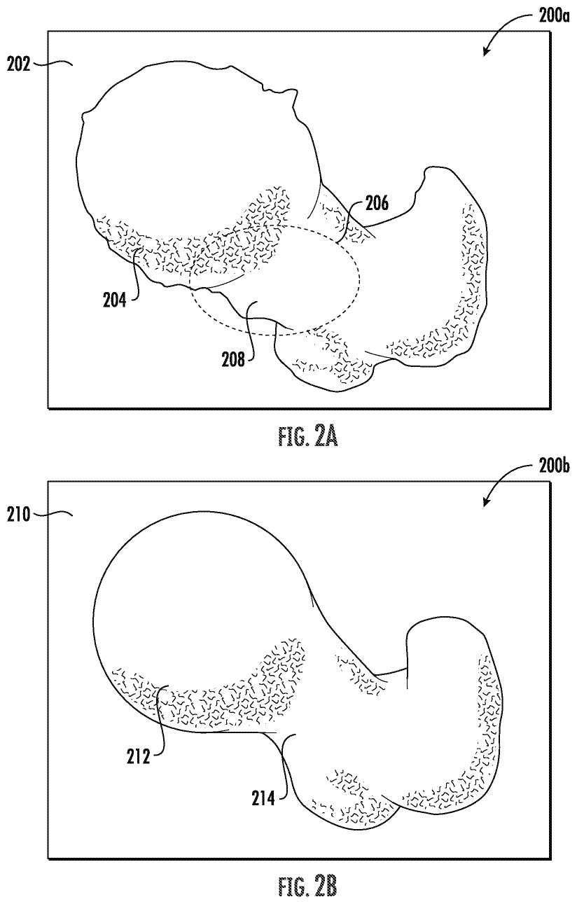

FIG. 2A and FIG. 2B illustrate examples of deformity of a three-dimensional (3D) pathological femur and proposed modifications, in accordance with non-limiting example(s) of the present disclosure. For example, FIG. 2A and FIG. 2B illustrate an example of 3D pathological proximal femur image 202 (shown in FIG. 2A) with deformed region(s) detected based on an inferred normalized bone image 210 (shown in FIG. 2B).

The 3D pathological proximal femur image 202 represents a CT scan of the proximal femur taken from a patient with femoroacetabular impingement (FAI). The inferred normalized bone image 210 can be generated by an ML model (e.g., inference model 118, or the like) from 3D pathological proximal femur image 202 as described herein. The inferred normalized bone image 210 can be registered onto the 3D pathological proximal femur image 202. Both the 3D pathological proximal femur image 202 and the inferred normalized bone image 210 can be partitioned and labeled. A segment of the 3D pathological proximal femur image 202 free of abnormality, such as the femur head 204, can be identified and matched to the corresponding femur head 212 of the inferred normalized bone image 210.

The remaining segments of the 3D pathological proximal femur image 202 can then be aligned to the respective remaining segments of the inferred normalized bone image 210. A comparison of the segments from the inferred normalized bone image 210 and the 3D pathological proximal femur image 202 reveals a region of deformity 206 on the femur neck 208 of the 3D pathological proximal femur image 202. The excess bone in the detected region of deformity 206 can be defined as the volumetric difference between the detected region of deformity 206 and the corresponding femur neck 214 on the femur neck 208. The volumetric difference can be used to form the basis of the surgical plan to define the shape and volume on the femur neck 208 that needs to be surgically removed.

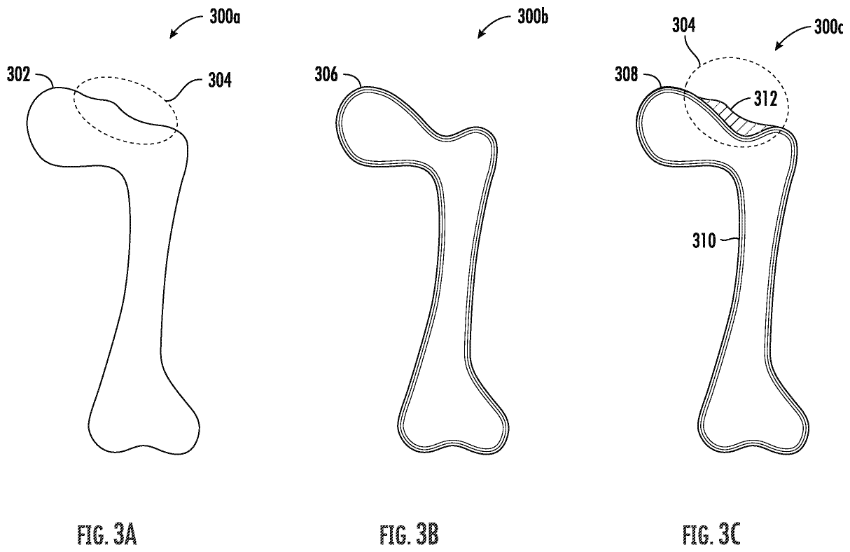

FIG. 3A, FIG. 3B, and FIG. 3C illustrate an example of a two-dimensional (2D) pathological femur and proposed modifications that can be derived using the present disclosure, in accordance with non-limiting example(s) of the present disclosure. For example, FIG. 3A illustrates 2D pathological femur image 300a depicting a femur 302 having a region of deformity 304.

Region of deformity 304 can be identified based on an inferred normalized bone image 300b depicting normalized femur 306 shown in FIG. 3B. The inferred normalized bone image 300b can be generated using an ML model (e.g., inference model 118, or the like) as described herein.

The inferred normalized bone image 300b can be registered to the 2D pathological femur image 300a to generate a registered femur model 308 shown in image 300c depicted in FIG. 3C. From the registered femur model 308, abnormality free region 310 (which can include one or more abnormality free segments) can be identified. By aligning the remaining segments of the registered femur model 308 to the corresponding segments of the femur 302 of the 2D pathological femur image 300a, region of deformity 304 is detected.

The region of deformity 304 defines the shape of the excess bone portion 312 on the femur 302 of the 2D pathological femur image 300a that may form the basis for a surgical plan.

The 3D example illustrates in FIG. 2A and FIG. 2B as well as the 2D example illustrated in FIG. 3A, FIG. 3B, and FIG. 3C are provided primarily to illustrate the concepts of abnormal bone image 120, normalized bone image 122, and surgery plan 124 described herein. Said differently, the example bone images depicted in these figures along with the regions of deformity are provided for purposes of clarity of explanation in describing inferring a normalized bone image from an ML model and generating a surgical plan based on the inferred normalized bone image.

FIG. 4 illustrates a logic flow 400, in accordance with non-limiting example(s) of the present disclosure. In general, logic flow 400 can be implemented by a system for removing portions of an abnormal bone or for generating a surgical plan for removing portions of an abnormal bone, such as, surgical planning system 100. Logic flow 400 is described with reference to surgical planning system 100 for purposes of clarity and description. Additionally, logic flow 400 is described with reference to the images and regions of deformity depicted in FIG. 2A and FIG. 2B as well as FIG. 3A to FIG. 3C. However, logic flow 400 could be performed by a system for generating a surgical plan for removing portions of an abnormal bone different than surgical planning system 100. Likewise, logic flow 400 can be used to generate a surgical plan for bones other than femur's or having deformities other than those depicted herein. Examples are not limited in this context.

Logic flow 400 can begin at block 402. At block 402 “receive a representation of an abnormal bone” a representation of an abnormal bone is received. At block 402, a computing device (e.g., computing device 102, or the like) can receive from an imaging device (e.g., imager 104, or the like) or from a memory device, data comprising indications of an abnormal bone. For example, processor 108 can execute instructions 116 to receive abnormal bone image 120. As a specific example, processor 108 can execute instructions 116 to receive abnormal bone image 120 from imager 104 or from a memory device storing abnormal bone image 120 (e.g., memory of imager 104, or another memory).

The represented abnormal bone can be a pathological bone undergoing surgical planning for alteration, repair, or removal. As noted above, the received abnormal bone image (e.g., abnormal bone image 120, or the like) can be data including a characterization of the abnormal bone. In an example, the data includes geometric characteristics including location, shape, contour, or appearance of the anatomical structure of the abnormal bone. In another example, the data includes intensity information (e.g., density, or the like) of the abnormal bone. Further, as noted above, the received abnormal bone image can include at least one medical image such as an X-ray, an ultrasound image, a computed tomography (CT) scan, a magnetic resonance (MR) image, a positron emission tomography (PET) image, a single-photon emission computed tomography (SPECT) image, or an arthrogram, among other 2D or 3D images.

Continuing to block 404 “infer, based on ML model, a representation of a normalized bone associated with the abnormal bone” a representation of a normalized bone associated with the abnormal bone is inferred from an ML model. In particular, at block 404, a representation of a normalized bone associated with the abnormal bone is inferred from an ML model. For example, computing device (e.g., computing device 102, or the like) can infer a representation of a normalized version of the abnormal bone represented by the representation received at block 402 from an ML model. As a specific example, processor 108 can execute instructions 116 and/or inference model 118 to infer normalized bone image 122 from abnormal bone image 120 and inference model 118.

As noted above, the inferred normalized bone image (e.g., normalized bone image 122, or the like) is data including a characterization of a desired postoperative shape or appearance of the abnormal bone. In an example, the data includes geometric characteristics including location, shape, contour, or appearance of the anatomical structure of the desired postoperative share or appearance of the abnormal bone. In another example, the data includes intensity information (e.g., density, or the like) of the desired post-operative abnormal bone. Further, as noted above, the inferred normalized bone image can include at least one medical image such as an X-ray, an ultrasound image, a computed tomography (CT) scan, a magnetic resonance (MR) image, a positron emission tomography (PET) image, a single-photon emission computed tomography (SPECT) image, or an arthrogram, among other 2D or 3D images.

As will be described in greater detail below, the inferred representation of the normalized bone associated with the abnormal bone image can be generated from an ML model trained to infer a normalized bone image from an abnormal bone image, where the ML model is trained with a data set including abnormal bone images and associated normalized bone images. These normalized bone images from the training data set can be medical images taken from normal bones of comparable anatomical origin from a group of subjects known to have normal bone anatomy and/or medical images taken from post-operative abnormal bones, or rather bones that have been normalized.

Continuing to block 406 “identify abnormal regions of the abnormal bone based on the normalized bone” abnormal regions (or an abnormal region) of the abnormal bone are identified from the normalized bone. In general, at block 406, processor 108 executes instructions 116 to compare, or match, the abnormal bone to the normal bone and differentiate the pathological portions of the abnormal bone from the non-pathological portions of the abnormal bone to identify regions of deformity on the abnormal bone. For example, processor 108 can execute instructions 116 to partition the abnormal bone and the normal bone into a number of segments representing various anatomical structures on the respective image. Processor 108 can further execute instructions 116 to label these segments such that the segments with the same label share specified characteristics such as a shape, anatomical structure, or intensity. Processor 108 can further execute instructions 116 to identify segments on the abnormal bone that do not have a corresponding label on the normalized bone as regions of deformity. In another example, processor 108 can execute instructions 116 to overlay the representation of the abnormal bone over the normalized bone and align the representations to identify areas of discontinuity in the representations.

With some embodiments, processor 108 can execute instructions 116 to extract features of the abnormal bone and the normalized bone in order to compare the bones and identify regions of deformity as described above. Extracted features can include geometric parameters such as a location, an orientation, a curvature, a contour, a shape, an area, a volume, or other geometric parameters. The extracted features can also include one or more intensity-based parameters.

In some embodiments, processor 108 can execute instructions 116 to determine a degree of similarity between the extracted features and/or segments of the abnormal bone and the normalized bone to determine whether the feature/segment is non-pathological or pathological. For example, processor 108 can execute instructions 116 to determine a degree of similarity based on distance in a normed vector space, a correlation coefficient, a ratio image uniformity, or the like. As another example, processor 108 can execute instructions 116 to determine a degree of similarity based on the type of the feature or a modality of the representation (e.g., CT image, X-ray image, or the like). For example, where the representation is 3D, the difference may be based on a volume.

Continuing to block 408 “generate a surgical plan for altering the abnormal bone based on the abnormal regions” a plan for modifying the abnormal bone based on the identified regions of deformity is generated. In general, at block 408, processor 108 executes instructions 116 to define a location, shape, and volume of a portion or portions of the abnormal bone from the one or more abnormal regions that need to be altered. For example, volumetric differences identified at block 406 can be flagged and coded for removal during surgery. Said differently, processor 108 can execute instructions 116 to identify areas of bone tissue in the abnormal bone to remove to “normalize” the abnormal bone. Such suggested modifications can be stored as surgery plan 124. In some embodiments, a graphic representation of the surgery plan 124 can be generated and displayed on I/O devices 112 of computing device 102. As a specific example, the portion of the bone flagged for removal can be color coded and displayed in the graphical representation.

With some embodiments, surgery plan 124 can include a first simulation of the abnormal bone and a second simulation of the surgically altered abnormal bone, such as a simulated model of the post-operative abnormal bone with the identified excess bone tissue removed. One or both of the first and the second simulations can each include a bio-mechanical simulation for evaluating one or more bio-mechanical parameters including, for example, range of motion of the respective bone.

In some embodiments, surgery plan 124 can include removal steps or removal passes to incrementally alter the abnormal region(s) of the abnormal bone by gradually removing the identified excess bone tissue from the abnormal bone. With some embodiments, a graphical user interface (GUI) element can be generated allowing input via I/O devices 112 to accept and/or modify the surgery plan 124.

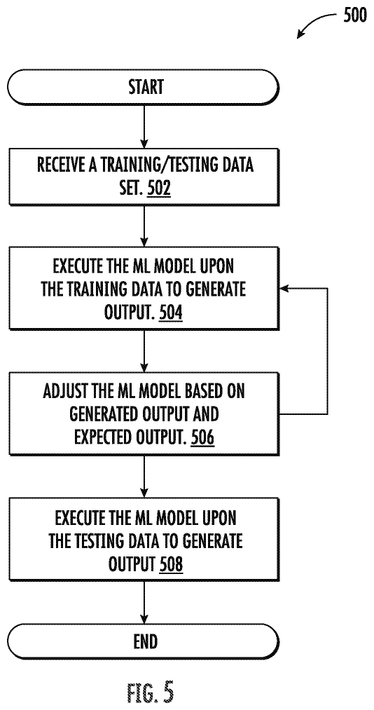

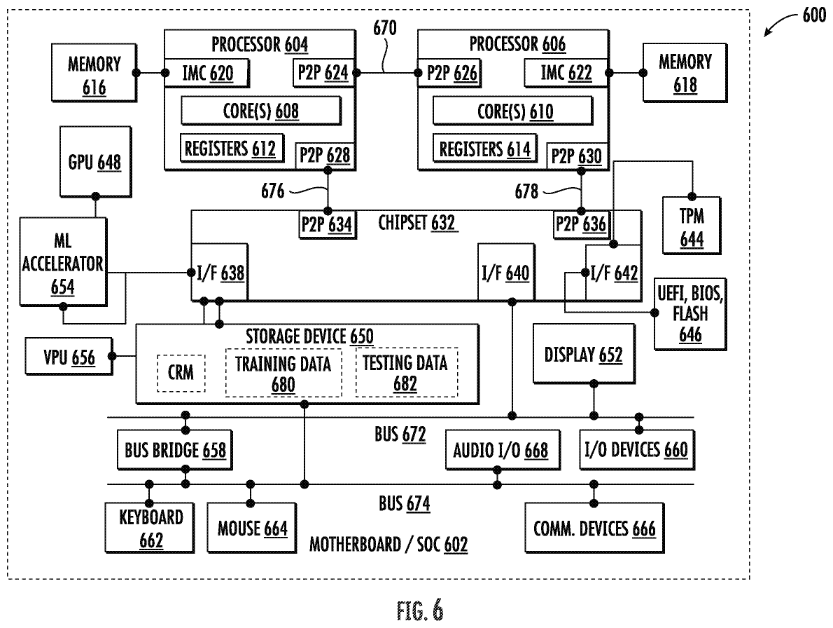

FIG. 5 illustrates a logic flow 500 for training and testing an ML model to infer a normalized bone image from an abnormal bone image, in accordance with non-limiting example(s) of the present disclosure. FIG. 6 describes a system 600. Logic flow 500 is described with reference to the system 600 of FIG. 6 for convenience and clarity. However, this is not intended to be limiting. In general, ML models are trained by an iterative process. Some examples of inference model training are given herein. However, it is noted that numerous examples provided herein can be implemented to train an ML model (e.g., inference model 118) independent on the algorithm(s) described herein.

Logic flow 500 can begin at block 502. At block 502 “receive a training/testing data set” a system can receive a training and testing data set. For example, system 600 can receive training data 680 and testing data 682. In general training data 680 and testing data 682 can comprise a number of pre-operative abnormal bone images and associated post-operative normalized bone images. In some embodiments, the collection of image pairs can be from procedures where the patient outcome was successful. With some embodiments, the pre-operative images include images modified based on a random pattern to simulate abnormalities found naturally within the populations bone anatomy.

In some embodiments, the images from training data 680 and testing data 682 can be pre-processed, for example, scaled, transformed, or modified to a common reference frame or plane. As a specific example, the training set images can be scaled to a common size and transformed to a common orientation in a geographic coordinate system. It is noted, that pre-processing during training/testing can be replicated during inference (e.g., at block 404, or the like).

With some embodiments, the images can include metadata or other characteristics or classifications, such as, bone type, age, gender, ethnicity, patient weight, patient height, surgery outcome, etc. In other embodiments, different testing and training sets, resulting in multiple trained ML models can be generated. For example, an ML model could be trained for gender specific inference, ethnic specific inference, or the like. In some embodiments, an ML model can be trained with multiple different bone types. In other embodiments, an ML model can be trained for a specific bone type. For example, training data 680 and testing data 682 could include only proximal femurs.

Continuing to block 504 “execute the ML upon the training data” the ML model is executed with the abnormal bone images from the training data 680 as input to generate an output. For example, processor 604/processor 606 can execute inference model 118 with the abnormal bone images from training data 680 as input to inference model 118. Continuing to block 506 “adjust the ML model based on the generated output and expected output” the ML model is adjusted based on the actual outputs from block 504 and the expected, or desired, outputs from the training set. For example, processor 604/processor 606 can adjust weights, connections, layers, or the like of inference model 118 based on the actual output at block 504 and the expected output. Often, block 504 and block 506 are iteratively repeated until inference model 118 converges upon an acceptable (e.g., greater than a threshold, or the like) success rate (often referred to as reaching a minimum error condition).

Continuing to block 508 “execute the ML model upon the testing data to generate output” the ML model is executed with the abnormal bone images from the testing data 682 as input to generate an output. For example, processor 604/processor 606 can execute inference model 118 with the abnormal bone images from testing data 682 as input to inference model 118. Furthermore, at block 508 processor 604/processor 606 can compare output from inference model 118 generated at block 508 with desired output from the testing data 682 to determine how well the ML model infers or generates correct output. With some examples, where the ML model does not infer testing data above a threshold level, the training set can be augmented and/or the ML model can be retrained, or training can be continued until the ML model infers untrained data above a threshold level.

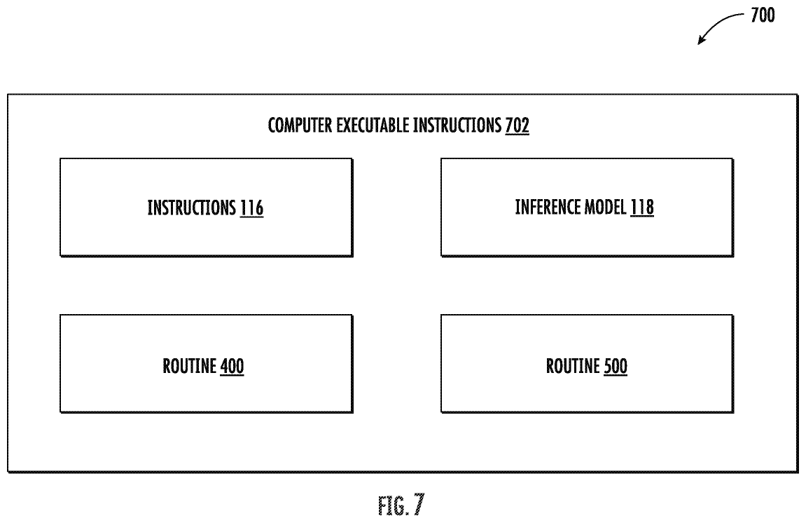

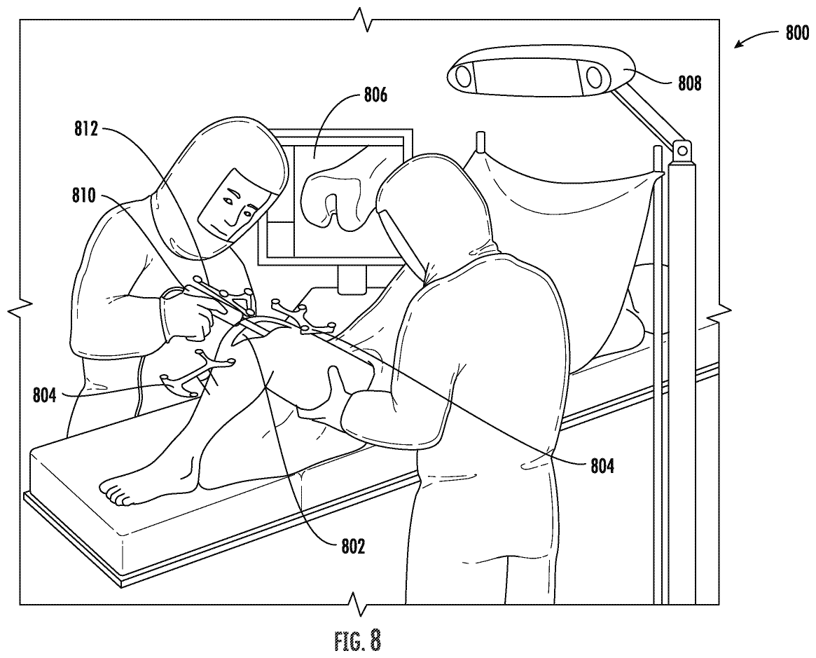

FIG. 6 illustrates an embodiment of a system 600. System 600 is a computer system with multiple processor cores such as a distributed computing system, supercomputer, high-performance computing system, computing cluster, mainframe computer, mini-computer, client-server system, personal computer (PC), workstation, server, portable computer, laptop computer, tablet computer, handheld device such as a personal digital assistant (PDA), or other device for processing, displaying, or transmitting information. Similar embodiments may comprise, e.g., entertainment devices such as a portable music player or a portable video player, a smart phone or other cellular phone, a telephone, a digital video camera, a digital still camera, an external storage device, or the like. Further embodiments implement larger scale server configurations. In other embodiments, the system 600 may have a single processor with one core or more than one processor. Note that the term “processor” refers to a processor with a single core or a processor package with multiple processor cores. In at least one embodiment, the computing system 600 is representative of the components of a computing system to train an ML model for use as described herein. In other embodiments, the computing system 600 is representative of components of computing device 102 or robotic surgical system 800. More generally, the computing system 600 is configured to implement all logic, systems, logic flows, methods, apparatuses, and functionality described herein with reference to FIG. 1, FIG. 4, FIG. 5, FIG. 7, and FIG. 8.

As used in this application, the terms “system” and “component” and “module” are intended to refer to a computer-related entity, either hardware, a combination of hardware and software, software, or software in execution, examples of which are provided by the exemplary system 600. For example, a component can be, but is not limited to being, a process running on a processor, a processor, a hard disk drive, multiple storage drives (of optical and/or magnetic storage medium), an object, an executable, a thread of execution, a program, and/or a computer. By way of illustration, both an application running on a server and the server can be a component. One or more components can reside within a process and/or thread of execution, and a component can be localized on one computer and/or distributed between two or more computers. Further, components may be communicatively coupled to each other by various types of communications media to coordinate operations. The coordination may involve the uni-directional or bi-directional exchange of information. For instance, the components may communicate information in the form of signals communicated over the communications media. The information can be implemented as signals allocated to various signal lines. In such allocations, each message is a signal. Further embodiments, however, may alternatively employ data messages. Such data messages may be sent across various connections. Exemplary connections include parallel interfaces, serial interfaces, and bus interfaces.

As shown in this figure, system 600 comprises a motherboard or system-on-chip (SoC) 602 for mounting platform components. Motherboard or system-on-chip (SoC) 602 is a point-to-point (P2P) interconnect platform that includes a first processor 604 and a second processor 606 coupled via a point-to-point interconnect 670 such as an Ultra Path Interconnect (UPI). In other embodiments, the system 600 may be of another bus architecture, such as a multi-drop bus. Furthermore, each of processor 604 and processor 606 may be processor packages with multiple processor cores including core(s) 608 and core(s) 610, respectively as well as registers including register(s) 612 and register(s) 614, respectively. While the system 600 is an example of a two-socket (2S) platform, other embodiments may include more than two sockets or one socket. For example, some embodiments may include a four-socket (4S) platform or an eight-socket (8S) platform. Each socket is a mount for a processor and may have a socket identifier. Note that the term platform refers to the motherboard with certain components mounted such as the processor 604 and chipset 632. Some platforms may include additional components and some platforms may only include sockets to mount the processors and/or the chipset. Furthermore, some platforms may not have sockets (e.g., SoC, or the like).

The processor 604 and processor 606 can be any of various commercially available processors, including without limitation an Intel® Celeron®, Core®, Core (2) Duo®, Itanium®, Pentium®, Xeon®, and XScale® processors; AMD® Athlon®, Duron® and Opteron® processors; ARM® application, embedded and secure processors; IBM® and Motorola® DragonBall® and PowerPC® processors; IBM and Sony® Cell processors; and similar processors. Dual microprocessors, multi-core processors, and other multi-processor architectures may also be employed as the processor 604 and/or processor 606. Additionally, the processor 604 need not be identical to processor 606.

Processor 604 includes an integrated memory controller (IMC) 620 and point-to-point (P2P) interface 624 and P2P interface 628. Similarly, the processor 606 includes an IMC 622 as well as P2P interface 626 and P2P interface 630. IMC 620 and IMC 622 couple the processors processor 604 and processor 606, respectively, to respective memories (e.g., memory 616 and memory 618). Memory 616 and memory 618 may be portions of the main memory (e.g., a dynamic random-access memory (DRAM)) for the platform such as double data rate type 3 (DDR3) or type 4 (DDR4) synchronous DRAM (SDRAM). In the present embodiment, the memories memory 616 and memory 618 locally attach to the respective processors (i.e., processor 604 and processor 606). In other embodiments, the main memory may couple with the processors via a bus and shared memory hub.

System 600 includes chipset 632 coupled to processor 604 and processor 606. Furthermore, chipset 632 can be coupled to storage device 650, for example, via an interface (I/F) 638. The I/F 638 may be, for example, a Peripheral Component Interconnect-enhanced (PCI-e). Storage device 650 can store instructions executable by circuitry of system 600 (e.g., processor 604, processor 606, GPU 648, ML accelerator 654, vision processing unit 656, or the like). For example, storage device 650 can store instructions for computer-readable storage media 700, training data 680, testing data 682, or the like.

Processor 604 couples to a chipset 632 via P2P interface 628 and P2P 634 while processor 606 couples to a chipset 632 via P2P interface 630 and P2P 636. Direct media interface (DMI) 676 and DMI 678 may couple the P2P interface 628 and the P2P 634 and the P2P interface 630 and P2P 636, respectively. DMI 676 and DMI 678 may be a high-speed interconnect that facilitates, e.g., eight Giga Transfers per second (GT/s) such as DMI 3.0. In other embodiments, the processor 604 and processor 606 may interconnect via a bus.

The chipset 632 may comprise a controller hub such as a platform controller hub (PCH). The chipset 632 may include a system clock to perform clocking functions and include interfaces for an I/O bus such as a universal serial bus (USB), peripheral component interconnects (PCIs), serial peripheral interconnects (SPIs), integrated interconnects (I2Cs), and the like, to facilitate connection of peripheral devices on the platform. In other embodiments, the chipset 632 may comprise more than one controller hub such as a chipset with a memory controller hub, a graphics controller hub, and an input/output (I/O) controller hub.

In the depicted example, chipset 632 couples with a trusted platform module (TPM) 644 and UEFI, BIOS, FLASH circuitry 646 via I/F 642. The TPM 644 is a dedicated microcontroller designed to secure hardware by integrating cryptographic keys into devices. The UEFI, BIOS, FLASH circuitry 646 may provide pre-boot code.

Furthermore, chipset 632 includes the I/F 638 to couple chipset 632 with a high-performance graphics engine, such as, graphics processing circuitry or a graphics processing unit (GPU) 648. In other embodiments, the system 600 may include a flexible display interface (FDI) (not shown) between the processor 604 and/or the processor 606 and the chipset 632. The FDI interconnects a graphics processor core in one or more of processor 604 and/or processor 606 with the chipset 632.

Additionally, ML accelerator 654 and/or vision processing unit 656 can be coupled to chipset 632 via I/F 638. ML accelerator 654 can be circuitry arranged to execute ML related operations (e.g., training, inference, etc.) for ML models. Likewise, vision processing unit 656 can be circuitry arranged to execute vision processing specific or related operations. In particular, ML accelerator 654 and/or vision processing unit 656 can be arranged to execute mathematical operations and/or operands useful for machine learning, neural network processing, artificial intelligence, vision processing, etc.

Various I/O devices 660 and display 652 couple to the bus 672, along with a bus bridge 658 which couples the bus 672 to a second bus 674 and an I/F 640 that connects the bus 672 with the chipset 632. In one embodiment, the second bus 674 may be a low pin count (LPC) bus. Various devices may couple to the second bus 674 including, for example, a keyboard 662, a mouse 664 and communication devices 666.

Furthermore, an audio I/O 668 may couple to second bus 674. Many of the I/O devices 660 and communication devices 666 may reside on the motherboard or system-on-chip (SoC) 602 while the keyboard 662 and the mouse 664 may be add-on peripherals. In other embodiments, some or all the I/O devices 660 and communication devices 666 are add-on peripherals and do not reside on the motherboard or system-on-chip (SoC) 602.

FIG. 7 illustrates computer-readable storage medium 700. Computer-readable storage medium 700 may comprise any non-transitory computer-readable storage medium or machine-readable storage medium, such as an optical, magnetic or semiconductor storage medium. In various embodiments, computer-readable storage medium 700 may comprise an article of manufacture. In some embodiments, 700 may store computer executable instructions 702 with which circuitry (e.g., processor 108, processor 604, processor 606, or the like) can execute. For example, computer executable instructions 702 can include instructions to implement operations described with respect to instructions 116, inference model 118, logic flow 400, and/or logic flow 500.

Examples of computer-readable storage medium 700 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 702 may include any suitable type of code, such as source code, compiled code, interpreted code, executable code, static code, dynamic code, object-oriented code, visual code, and the like.

FIG. 8 illustrates a robotic surgical system 800, in accordance with non-limiting example(s) of the present disclosure. In general, robotic surgical system 800 is for performing an orthopedic surgical procedure using a robotic system (e.g., surgical navigation system, or the like). Robotic surgical system 800 includes a surgical cutting tool 810 with an associated optical tracking frame 812 (also referred to as tracking array), graphical user interface (GUI) 806, an optical tracking system 808, and patient tracking frames 804 (also referred to as tracking arrays). In some embodiments, surgical tool 106 of surgical planning system 100 of FIG. 1 can be the surgical cutting tool 810 and associated patient tracking frame 804, optical tracking frame 812, and optical tracking system 808 while the GUI 806 can be provided on a display (e.g., I/O devices 112 of computing device 102 of surgical planning system 100 of FIG. 1).

This figure further depicts an incision 802, through which a knee revision surgery may be performed. In an example, the illustrated robotic surgical system 800 depicts a hand-held computer-controlled surgical robotic system. The illustrated robotic system uses optical tracking system 808 coupled to a robotic controller (e.g., computing device 102, or the like) to track and control a hand-held surgical instrument (e.g., surgical cutting tool 810). For example, the optical tracking system 808 tracks the optical tracking frame 812 coupled to the surgical cutting tool 810 and patient tracking frame 804 coupled to the patient to track locations of the instrument relative to the target bone (e.g., femur and tibia for knee procedures).

By using genuine models of anatomy more accurate surgical plans may be developed than through statistical modeling.

The following examples pertain to further embodiments, from which numerous permutations and configurations will be apparent.

Example 1. A method comprising: receiving, at a computing device, a representation of an abnormal bone; inferring a representation of a normalized bone associated with the abnormal bone based on executing a machine learning (ML) model at the computing device with the representation of the abnormal bone as input to the ML model; identifying a region of deformity on the abnormal bone based on the representation of the normalized bone; and generating a surgical plan for altering the abnormal bone based on the region of deformity.

Example 2. The method of example 1, comprising: partitioning the abnormal bone into a plurality of segments; partitioning the normalized bone into a plurality of segments; and identifying from the segments of the abnormal bone the region of deformity.

Example 3. The method of any one of examples 1 to 2, comprising: extracting a first plurality of anatomical features from the abnormal bone; extracting a second plurality of anatomical features from the normalized bone; comparing the first plurality of features to the second plurality of features to identify the region of deformity.

Example 4. The method of any one of examples 1 to 3, wherein the ML model comprises a convolutional neural network (CNN).

Example 5. The method of any one of examples 1 to 4, wherein the ML model is trained with a data set comprising a plurality of images of pathological bones and for each one of the plurality of images of the pathological bones, an associated image of a non-pathological bone.

Example 6. The method of example 5, wherein at least one of the associated image of the non-pathological bone is of a post-operative pathological bone.

Example 7. The method of any one of examples 5 or 6, wherein the plurality of images of the pathological bones are classified as having at least one of the same bone type, the same gender assigned at birth, the same ethnicity, or the same age range.

Example 8. The method of any one of examples 1 to 7, wherein the bone type is a femur.

Example 9. The method of any one of examples 1 to 8, comprising generating control signals for a surgical tool of a surgical navigation system based on the surgical plan.

Example 10. A non-transitory computer-readable storage medium, the computer-readable storage medium including instructions that when executed by a computer, cause the computer to: receive, at a computing device, a representation of an abnormal bone; infer a representation of a normalized bone associated with the abnormal bone based on executing a machine learning (ML) model at the computing device with the representation of the abnormal bone as input to the ML model; identify a region of deformity on the abnormal bone based on the representation of the normalized bone; and generate a surgical plan for altering the abnormal bone based on the region of deformity.

Example 11. The computer-readable storage medium of example 10, comprising instructions that when executed by the computer cause the computer to: partition the abnormal bone into a plurality of segments; partition the normalized bone into a plurality of segments; and identify from the segments of the abnormal bone the region of deformity.

Example 12. The computer-readable storage medium of any one of examples 10 to 11, comprising instructions that when executed by the computer cause the computer to: extract a first plurality of anatomical features from the abnormal bone; extract a second plurality of anatomical features from the normalized bone; compare the first plurality of features to the second plurality of features to identify the region of deformity.

Example 13. The computer-readable storage medium of any one of examples 10 to 12, wherein the ML model comprises a convolutional neural network (CNN).

Example 14. The computer-readable storage medium of any one of examples 10 to 13, wherein the ML model is trained with a data set comprising a plurality of images of pathological bones and for each one of the plurality of images of the pathological bones, an associated image of a non-pathological bone.

Example 15. The computer-readable storage medium of example 14, wherein at least one of the associated image of the non-pathological bone is of a post-operative pathological bone.

Example 16. The computer-readable storage medium of any one of examples 14 or 15, wherein the plurality of images of the pathological bones are classified as having at least one of the same bone type, the same gender assigned at birth, the same ethnicity, or the same age range.

Example 17. The computer-readable storage medium of any one of examples 10 to 16, wherein the bone type is a femur.

Example 18. The computer-readable storage medium of any one of examples 10 to 17, comprising generate control signals for a surgical tool of a surgical navigation system based on the surgical plan.

Example 19. A computing apparatus comprising: a processor; and a memory storing instructions that, when executed by the processor, configure the apparatus to: receive, at a computing device, a representation of an abnormal bone; infer a representation of a normalized bone associated with the abnormal bone based on executing a machine learning (ML) model at the computing device with the representation of the abnormal bone as input to the ML model; identify a region of deformity on the abnormal bone based on the representation of the normalized bone; and generate a surgical plan for altering the abnormal bone based on the region of deformity.

Example 20. The computing apparatus of example 19, the memory storing instructions that, when executed by the processor, configure the apparatus to: partition the abnormal bone into a plurality of segments; partition the normalized bone into a plurality of segments; and identify from the segments of the abnormal bone the region of deformity.

Example 21. The computing apparatus of any one of example 19 to 20, the memory storing instructions that, when executed by the processor, configure the apparatus to: extract a first plurality of anatomical features from the abnormal bone; extract a second plurality of anatomical features from the normalized bone; compare the first plurality of features to the second plurality of features to identify the region of deformity.

Example 22. The computing apparatus of any one of examples 19 to 21, wherein the ML model comprises a convolutional neural network (CNN).

Example 23. The computing apparatus of any one of examples 19 to 22, wherein the ML model is trained with a data set comprising a plurality of images of pathological bones and for each one of the plurality of images of the pathological bones, an associated image of a non-pathological bone.

Example 24. The computing apparatus of example 23, wherein at least one of the associated image of the non-pathological bone is of a post-operative pathological bone.

Example 25. The computing apparatus of any one of examples 23 or 24, wherein the plurality of images of the pathological bones are classified as having at least one of the same bone type, the same gender assigned at birth, the same ethnicity, or the same age range.

Example 26. The computing apparatus of any one of examples 19 to 25, wherein the bone type is a femur.

Example 27. The computing apparatus of any one of examples 19 to 26, comprising generate control signals for a surgical tool of a surgical navigation system based on the surgical plan.

Example 28. A surgical navigation system, comprising: a surgical cutting tool; and the computing apparatus of any one of examples 19 to 27 coupled to the surgical cutting tool, wherein the control signals are for the surgical cutting tool.

1. A method comprising:
receiving, at a computing device, a representation of an abnormal bone;
inferring a representation of a normalized bone associated with the abnormal bone based on executing a machine learning (ML) model at the computing device with the representation of the abnormal bone as input to the ML model;
identifying a region of deformity on the abnormal bone based on the representation of the normalized bone; and
generating a surgical plan for altering the abnormal bone based on the region of deformity.

2. The method of claim 1, comprising:
partitioning the abnormal bone into a plurality of segments;
identifying the region of deformity based on the plurality of segments of the abnormal bone.

3. The method of claim 2, comprising:
partitioning the normalized bone into a plurality of segments; and
identifying the region of deformity based on the plurality of segments of the abnormal bone and the plurality of segments of the normalized bone.

4. The method of claim 1, comprising:
comparing a first plurality of anatomical features associated with the abnormal bone with a second plurality of anatomical features associated with the normalized bone; and
identifying the region of deformity based on the comparison of the first plurality of anatomical features with the second plurality of anatomical features.

5. The method of claim 4, comprising:
extracting the first plurality of anatomical features from the representation of the abnormal bone; and
extracting the second plurality of anatomical features from the representation of the normalized bone.

6. The method of claim 1, wherein the ML model comprises a convolutional neural network (CNN).

7. The method of claim 1, wherein the ML model is trained with a data set comprising a plurality of images of pathological bones and for each one of the plurality of images of the pathological bones, an associated image of a non-pathological bone.

8. The method of claim 7, wherein at least one of the plurality of associated images of the non-pathological bone is of a post-operative pathological bone.

9. The method of claim 7, wherein at least one of the plurality of associated images of the non-pathological bone is a one of the plurality of images of pathological bones comprising at least one randomly generated anatomical feature.

10. The method of claim 7, wherein the plurality of images of the pathological bones are classified as having at least one of the same bone type, the same gender assigned at birth, the same ethnicity, or the same age range.

11. The method of claim 7, wherein the plurality of images of the pathological bones are classified as having a surgical outcome.

12. The method of claim 1, wherein the bone type is a femur.

13. The method of claim 1, comprising generating control signals for a surgical tool of a surgical navigation system based on the surgical plan.

14. A non-transitory computer-readable storage medium, the computer-readable storage medium including instructions that when executed by a computer, cause the computer to perform the method of claim 1.

15. A surgical navigation system, comprising:
a surgical cutting tool; and
a computing apparatus comprising a processor and memory comprising instructions that when executed by the processor cause the processor to perform the method of claim 1.
